(19) United States
(12) Patent Application Publication
(10) Pub. No.: US 2021/0095049 A1
(43) Pub. Date: Apr. 1, 2021

(54) METHOD OF INHIBITING ECTOPIC CALCIFICATION

(71) Applicant: ConcieValve LLC, Minneapolis, MN (US)

(72) Inventor: Nalini M. Rajamannan, Chicago, IL (US)

(21) Appl. No.: 16/876,350

(22) Filed: May 18, 2020

Related U.S. Application Data
(63) Continuation of application No. 15/874,186, filed on Jan. 18, 2018, now abandoned.

(51) Int. Cl. C07K 16/40; A61K 9/00; A61P 3/00; A61K 31/00

(52) U.S. Cl. CPC C07K 16/40; A61K 9/0019; C07K 2317/21; A61K 31/00; A61P 3/00

(57) ABSTRACT

A method for inhibiting ectopic calcification of bodily organs caused by elevated LDL cholesterol is provided. The method includes administering an LDL lowering agent to a patient, comprising a PCSK9 inhibitor selected from Evolocumab, Alirocumab, Praluent, Repatha Pushtonix, Repatha autoinjector, and combinations of the foregoing.

### CROSS-REFERENCE TO RELATED APPLICATION(S)

This application is a continuation of U.S. Ser. No. 15/874,186, filed Jan. 18, 2018, the entire contents of which are hereby incorporated by reference.

### FIELD OF THE INVENTION

The invention relates to the abnormal development of ectopic calcification in organs, tissue, vasculature and skeleton. In particular, the invention relates to a method of inhibiting ectopic calcification in the human body including the heart, the skeleton, the spinal column, the spine, the joints, the eyes, the brain, the kidneys, the GI tract, heart valves, and the vasculature.

### BACKGROUND OF THE INVENTION

Ectopic calcification is the formation of bone deposits in abnormal places in the human body. For example, the heart is a hollow, muscular organ that circulates blood throughout and animal's body by contracting rhythmically. In mammals, the heart has four-chambers situated such that the right atrium and ventricle are completely separated from the left atrium and ventricle. Normally, blood flows from systemic veins to the right atrium, and then to the right ventricle from which it is driven to the lungs via the pulmonary artery. Upon return from the lungs, the blood enters the left atrium, and then flows to the left ventricle from which it is driven into the systemic arteries.

Four main heart valves prevent the backflow of blood during the rhythmic contractions: the tricuspid, pulmonic valve, mitral and aortic valves. The tricuspid valve separates the right atrium and right ventricle, the pulmonary valve separates the right atrium and pulmonary artery, the mitral valve separates the left atrium and left ventricle, and the aortic valve separates the left ventricle and aorta.

The skeleton including the long bones, the joints, the spine, is the internal structure of vertebrate animals, comprising bone and cartilage, that supports the body and serves as a framework for the attachment of muscles, and protects the vital organs and associated structures.

The complex human brain controls all major body functions as well as how an individual feel, acts and thinks. The brain is housed in the skull, which protects it from injury. The spinal column protects the spinal cord, nerve roots and several of the body's internal organs. It also provides structural support and balance to maintain an upright posture and enables flexible motion.

The kidneys regulate fluid balance in the body and filter out waste from the blood in the form of urine.

The gastrointestinal tract is an organ system within humans and other animals which takes in food, digests it to extract and absorb energy and nutrients, and expels the remaining waste as feces.

The eye is defined as the Transparent front segment of the eye that covers iris, pupil, and anterior chamber, and provides most of an eye's optical power via the lens.

The heart, brain, skeleton and spinal column can malfunction by forming ectopic calcification thereby causing severe deleterious effects to an individual. For example, the abnormal calcification of the spinal column may cause limitations in walking and movement. Calcification in the vasculature may result in obstructions of blood flow. Calcification in the skeleton may cause osteoarthritis. Abnormal calcification in the brain may cause memory loss. Abnormal calcification in the kidneys may cause renal disease and glomerulonephritis. Abnormal calcification in the GI tract may cause polyps and cancer. Abnormal calcification in the intraocular lens may cause cataracts.

Therefore, what is needed is a method of inhibiting ectopic calcification in the human body.

### BRIEF SUMMARY OF THE INVENTION

The foregoing need is addressed by the method in accordance with the invention.

A method for inhibiting ectopic calcification in bodily organs caused by elevated LDL cholesterol levels is provided. The method includes administering an LDL lowering medication to a patient, wherein the LDL lowering medication may be a PCSK9 inhibitor selected from Evolocumab, Alirocumab, Praluent, Repatha Pushtonix, Repatha autoinjector, and combinations of the foregoing; inhibiting the activation of Lrp5-TIEG pathways; and slowing or reversing the progression of ectopic calcification caused by elevated LDL levels in the bloodstream, which activates the Lrp5 receptor. The method further includes inhibiting extracellular protein matrix production in an osteoblast cell originating from a native myofibroblast cell.

A subcutaneous dosage of the LDL lowering medication and/or the PCSK9 inhibitor may be administered subcutaneously or intramuscularly.

An initial dose of the PCSK9 inhibitor is from 0.25 mg/kg to 1.5 mg/kg.

### BRIEF DESCRIPTION OF THE DRAWINGS

For a better understanding of the invention, and to show how the same may be carried into effect, reference will now be made, by way of example, to the accompanying drawings, in which:

FIG. 1 is graphical illustration showing the decrease in LDL cholesterol after six months of PCSK9 therapy compared to the same levels three years prior.

FIG. 2 is a graphical illustration showing the improvement in the Echo Parameter Mid Aorta measurement after six months of PCSK9 therapy compared to the same levels three years prior.

FIG. 3 is a graphical representation showing the improvement of regurgitant proximal flow velocity in a patient after treatment for six months with PCSK9 therapy compared to the same levels three years prior.

FIG. 4 is a graphical representation showing the improvement in the echo measurement of the regurgitant after six months of PCSK9 therapy compared to the same levels three years prior.

FIG. 5 illustrates the role of Lrp5-TIEG1 in ectopic calcification.

FIG. 6 is a phenotype characterization of mouse knee joints on a cholesterol diet with and without atorvastatin therapy.

FIG. 7 is a phenotype characterization of mouse kidneys on a cholesterol diet with and without atorvastatin therapy.

FIG. 8 illustrates the histology of brains removed from LDLR knockout mice on three different diets, control cholesterol and cholesterol plus atorvastatin

### DETAILED DESCRIPTION OF THE INVENTION

The invention involves methods and material related to the development of ectopic calcification in the human body including heart valves, spinal column, brain, kidneys, gastrointestinal tract and eyes which can cause malfunction of all of these organs. The invention provides a treatment to inhibit the formation of ectopic calcification in these organs which is caused by elevated LDL which activates the Lrp5 pathway in these organs to form abnormal ectopic bone formation in these organs. The calcification which develops in these organs causes malfunction of the specific organs in the human body. The invention provides methods and materials for (1) slowing heart valve degeneration, and calcification, (2) treating carcinoid heart disease, (3) slowing progression of spinal stenosis secondary to ectopic calcification, (4) slowing the progression of renal disease secondary to ectopic bone formation, (5) slowing the progression of GI abnormalities secondary to abnormal Lrp5 activation and expression of extracellular matrix formation, (6) slowing the progression of osteoarthritis secondary to abnormal extracellular material proteins in the joints, (7) slowing the progression of cataract formation secondary to abnormal calcification in the lens, (8) slowing the progression of rheumatic valve disease secondary to ectopic calcification in the rheumatic valves. To date, there are no randomized clinical trials in humans which have proven whether lipid lowering can slow the progression of ectopic calcification in these various organs. This invention will inhibit the PCSK9 receptor to slow the progression of the calcification by lowering LDL cholesterol levels.

The invention is based on the discovery that including heart valves, spinal column, brain, kidneys, gastrointestinal tract and eyes express abnormal bone matrix proteins which cause heart valve calcification via the Lrp5-TIEG1 pathway. Specifically, the role of LDL activation of Lrp5 complex binding to Wnt, Frizzled, to activate β-catenin, TIEG1, and LEF1 in the nucleus. The activation of these signaling pathway critical in ectopic abnormal bone formation in the human body in different organs.

The invention is also based on the discovery that ectopic calcification can develop in heart valves, spinal column, brain, kidneys, gastrointestinal tract and eyes secondary to elevated LDL causing long term ectopic calcification and that inhibiting the level of LDL in the body can treat the disease mechanisms causing calcification including the use of PCSK9 inhibitors.

### Methods

ApoE^{-/-}/Lrp5 experimental hypercholesterolemia mouse model

ApoE^{-/-} mice were purchased from Jackson Laboratories (Bar Harbor, Me.) and Lrp5^{-/-} were purchased from Taconic laboratories (Germantown, N.Y.). ApoE^{-/-}:Lrp5^{-/-} were produced by cross breeding. Mice aged 6-8 weeks (male and female mice) were assigned to a control (N=60), a 0.2% cholesterol (w/w) diet (Harlan Teklad 88137), (N=60) and a 0.2% cholesterol (w/w) diet (Harlan Teklad 88137). All animals were fed ad libitum for 23 weeks. Control mice were fed a standard diet. Following this 23-week period, the mice were euthanized with inhalation CO₂. All experiments were performed in an animal facility accredited by the Association for Assessment and Accreditation of Laboratory Animal Care, Inc. (ACUC-A3283-01, 1-08-382). Immediately after dissection from the heart one leaflet from each aortic valve was fixed in 10% buffered formalin for 24 hours and then embedded in paraffin. Valves were also snap frozen in liquid nitrogen and stored in −80 degree freezer for gene expression experiments. RealTime PCR was performed to measure Lrp6, TIEG1 and Runx2 in the cardiac valves.

### Cell Lines and Culture Conditions

Valve Interstitial cells were isolated as described previously. Briefly, the valves were dissected out, cleaned of all tissue, rinsed in PBS, and minced. Valves were subjected to 3 collagenase digestions and interstitial cells were isolated from the third digestion were plated in α-MEM (Invitrogen, Carlsbad, Calif.) containing 10% fetal bovine serum (FBS) (Gemini Bio-Products, West Sacramento, Calif.) and 1% antibiotic/antimycotic (ThermoFisher Scientific, Waltham, Mass.) and propagated in a humidified incubator with 5% CO₂. ANOVA statistical analysis will be performed to test the differences in the different treatment groups (A p value of less than 0.05 is significant.)

### Transient Transfection and Luciferase Assays

Valve interstitial cells were plated at a density of 50% in 12 well plates in replicates of 6. As indicated, cells were transfected with 250 ng of the TOP FLASH reporter and/or various expression vectors (empty pcDNA4.0, Flag-tagged TIEG1, Flag-tagged Lef1, constitutively active β-catenin or Xpress-tagged TIEG1 domain expression constructs using Fugene-6 (Roche, Indianapolis, Ind.) as specified by the manufacturer. Empty vector was added to transfections as necessary to normalize the total amount of DNA transfected across each condition. Twenty-four hours following transfection, cells were lysed in passive lysis buffer (Promega, Madison, Wis.), lysates were quantitated for protein content, and equal amounts of protein were used to measure luciferase activity using Luciferase Assay Reagent (Promega) and a Glomax-Dual luminometer (Promega).

### Confocal Microscopy

Valvular interstitial cells were plated on coverslips at low confluence and allowed to adhere overnight. Cells were transfected as indicated or treated with TGFβ (2 ng/mL) for 24 hours. Cells were fixed in 1% paraformaldehyde for 30 min and washed twice with 1×PBS followed by permeabilization with 0.2% Triton-X in PBS for 30 min and blocked for an additional 30 min in heat-inactivated 5% FBS. Subsequently, cells were incubated with a polyclonal TIEG1 antibody and a monoclonal β-catenin antibody (clone 14/beta-catenin (RUO)) for 60 min. Cells were washed twice with PBS and stained with Texas Red- and FITC-conjugated secondary IgG Antibodies (Santa Cruz Biotechnology, Santa Cruz, Calif.) for an additional 60 minutes. DAPI was used as a counterstain. Immunofluorescence images were captured with a Zeiss LSM 510 confocal microscope (Carl Zeiss, Jena, Germany).

### Results

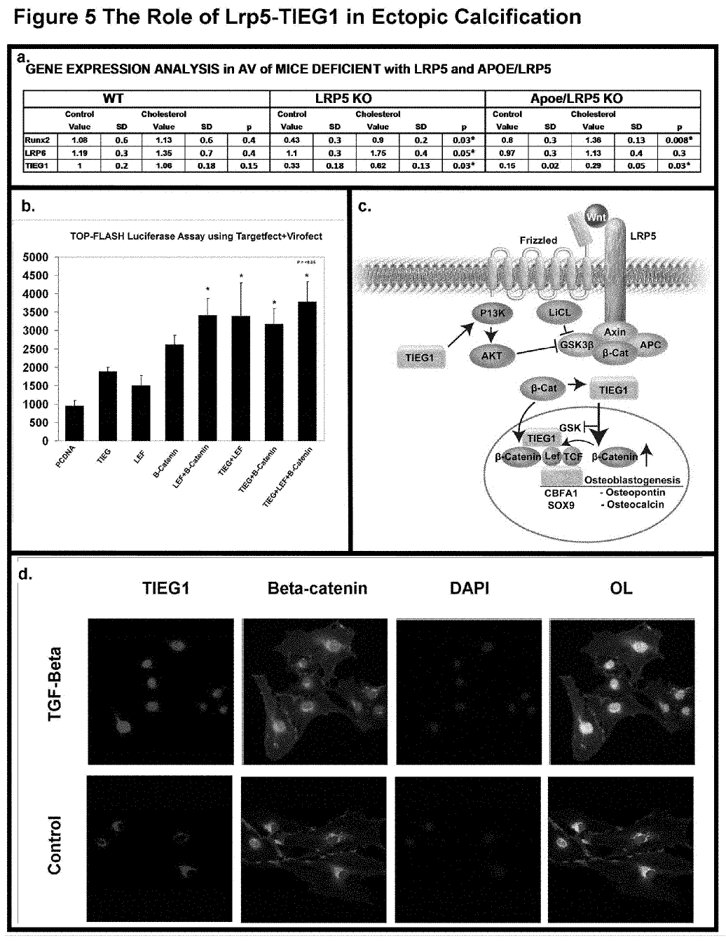

To understand if Lrp5^{-/-}/ApoE^{-/-}, hypercholesterolemic aortic valves osteogenesis is regulated via upregulation of TIEG1 expression we performed RTPCR on the valves from our established model of valve osteogenesis. FIG. 5, Panel a, is the quantification of the gene expression for the hypercholesterolemic wildtype valves versus the hypercholesterolemic Lrp5^{-/-} valves, ApoE^{-/-}:Lrp5^{-/-} valves. The real time PCR confirmed an upregulation of TIEG1, Runx2, in the Lrp5^{-/-} valves and double knockout ApoE^{-/-}:Lrp5^{-/-}, and again a significant increase in Lrp6 in the Lrp5^{-/-} valves with a mild increase in the Lrp6 in the ApoE^{-/-}:Lrp5^{-/-} valves hypercholesterolemic aortic valve. This current study demonstrates an increase in the TIEG1/Runx2/Lrp6 gene expression in the hypercholesterolemic aortic valves from the single and double knock out mice as compared to the WT control mice, confirming the role of elevated lipids in the osteogenic gene cascade, with the novel finding of TIEG1 upregulation in these valves. Co-expression of both β-catenin and LEF1 led to co-activation of the top-flash reporter when transfected into VICs. When TEG1 was co-expressed with LEF or β-catenin, there was a significant increase in the reporter activity was observed. These data suggested that TIEG regulates, LEF and β-catenin to form a transcriptionally active protein complex leading to enhanced Wnt signaling in VICs as shown in FIG. 5, Panel b. FIG. 5, Panel c, outlines the signaling pathway in valve osteogenesis via Lrp5/6/TIEG1 signaling. This possibility was further confirmed by the observation that TIEG1 and β-catenin co-localize with one another in the nucleus of VICs following stimulation with TGF-β treatment, a known regulator of TIEG1 expression as shown in FIG. 5, Panel d.

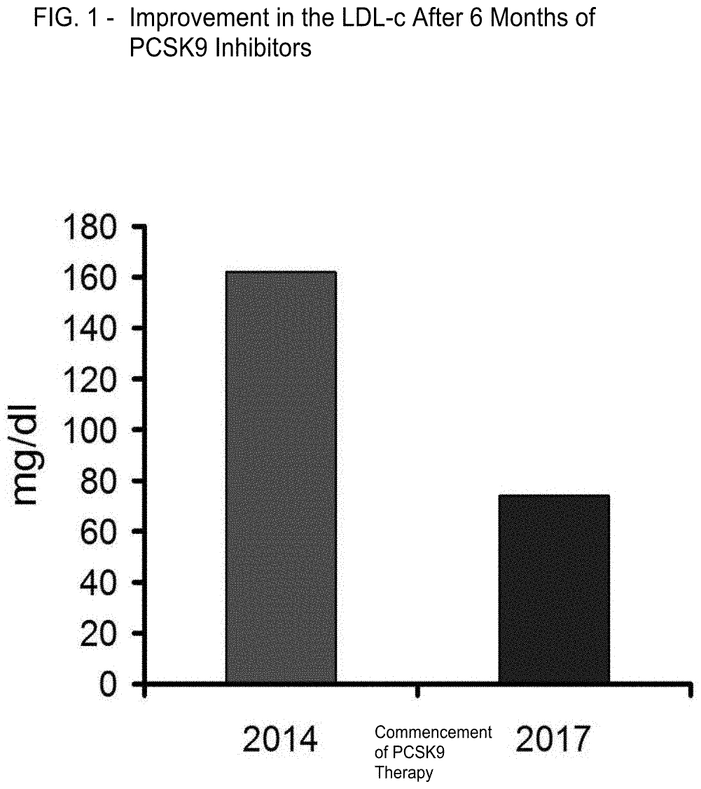

Referring now to the FIGS., FIG. 1 demonstrates the decrease in LDL cholesterol of a patient after treatment with a biweekly injection of a PCSK9 inhibitor for 6 months in a patient compared to the LDL cholesterol of the patient three years prior.

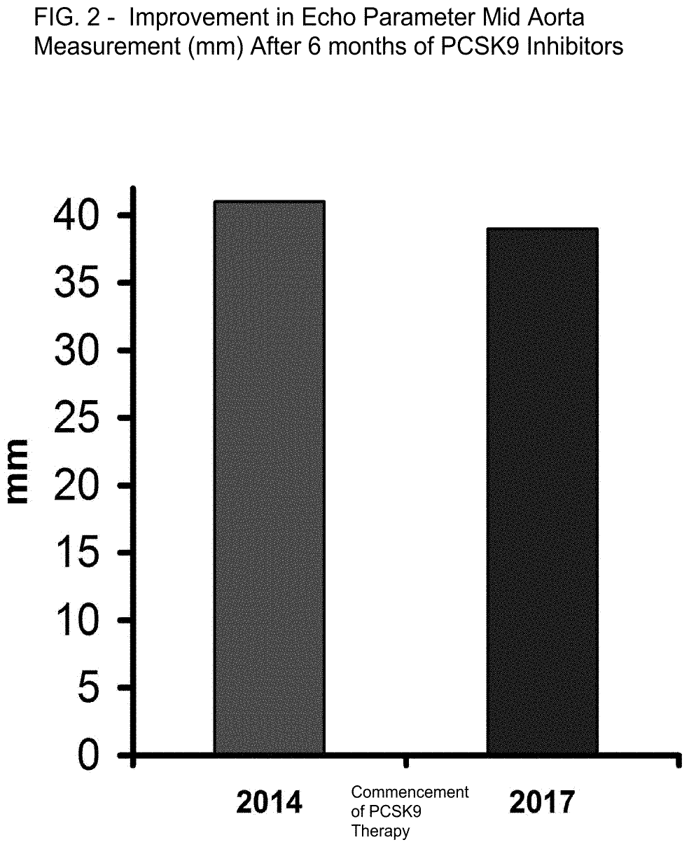

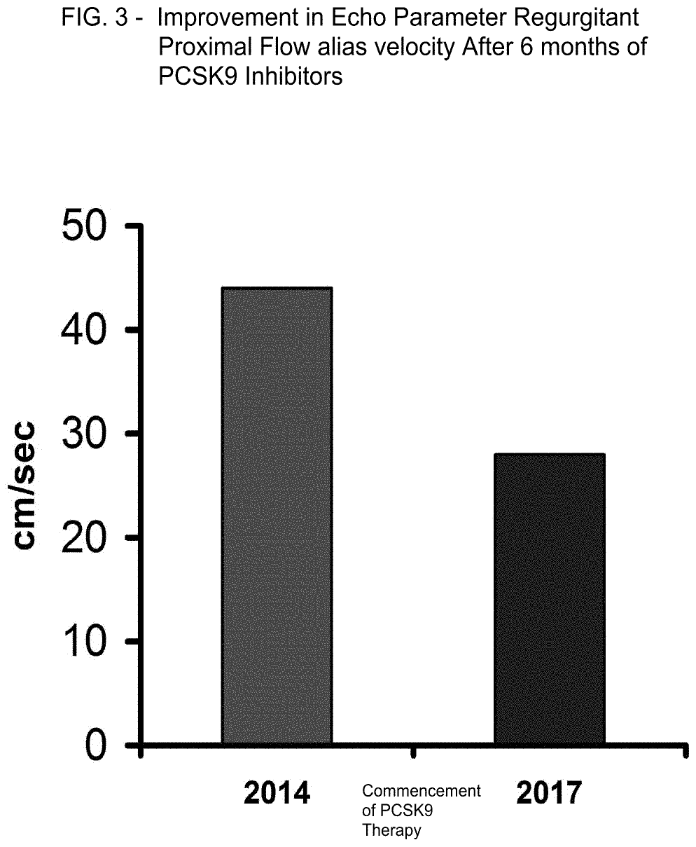

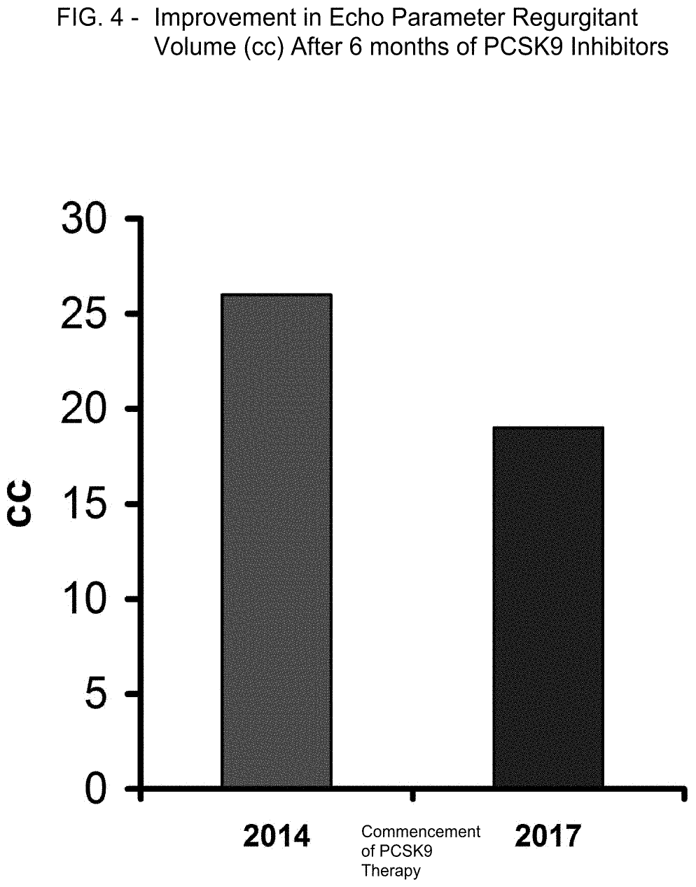

FIG. 2 shows the improvement in the echo parameter mid-aorta measurement in a patient after 6 months of PCSK9 therapy compared to the same measurement made three years prior. FIG. 3 is a bar graph plotting the improvement of the regurgitant proximal flow velocity in a patient after treatment for 6 months with a PCSK9 inhibitor compared to the same measurement three years prior. FIG. 4 is a graphical illustration showing the improvement in the echo measurement of the regurgitant volume of a patient after 6 months of a PCSK9 inhibitor compared to the same measurement three years prior.

FIG. 5 illustrates the Role of Lrp5-TIEG1 in ectopic calcification. FIG. 5, Panel a, demonstrates the RT PCR results for the ApoE^{-/-}:Lrp5^{-/-} valves versus WT control mice on hypercholesterolemic diets. FIG. 5, Panel b, demonstrates the regulation of TOPFLASH reporter after transfection with TIEG1, LEF and b-catenin. FIG. 5, Panel c, demonstrates the role of TIEG1 in aortic valve osteogenesis via Wnt Signaling. FIG. 5, Panel d, demonstrates the confocal microscopy of TIEG1 translocation to the nucleus in the presence of TGF-beta. To understand the development of ectopic calcification in the heart, Lrp5^{-/-}/ApoE^{-/-}, hypercholesterolemic aortic valves osteogenesis is regulated via upregulation of TIEG1 expression the inventor performed RTPCR on the valves from her established model of valve osteogenesis. FIG. 5, Panel a, is the quantification of the gene expression for the hypercholesterolemic wild type valves versus the hypercholesterolemic Lrp5^{-/-} valves, ApoE^{-/-}:Lrp5^{-/-} valves. The real time PCR confirmed an upregulation of TIEG1, Runx2, in the Lrp5^{-/-} valves and double knockout ApoE^{-/-}:Lrp5^{-/-}, and again a significant increase in Lrp6 in the Lrp5^{-/-} valves with a mild increase in the Lrp6 in the ApoE^{-/-}:Lrp5^{-/-} valves hypercholesterolemic aortic valve. This demonstrates an increase in the TIEG1/Runx2/Lrp6 gene expression in the hypercholesterolemic aortic valves from the single and double knock out mice as compared to the WT control mice, confirming the role of elevated lipids in the osteogenic gene cascade, with the novel finding of TIEG1 upregulation in these valves. Co-expression of both β-catenin and LEF1 led to co-activation of the top-flash reporter when transfected into VICs. When TIEG1 was co-expressed with LEF or β-catenin, a significant increase in the reporter activity was observed. This data suggests that TIEG1 regulates LEF and β-catenin to form a transcriptionally active protein complex leading to enhanced Wnt signaling in VICs as shown in FIG. 5, Panel b. FIG. 5, Panel c, outlines the signaling pathway in valve osteogenesis via Lrp5/6/TIEG1 signaling. The foregoing was further confirmed by the observation that TIEG1 and β-catenin co-localize with one another in the nucleus of VICs following stimulation with TGF-β treatment, a known regulator of TIEG1 expression as shown in FIG. 5, Panel d.

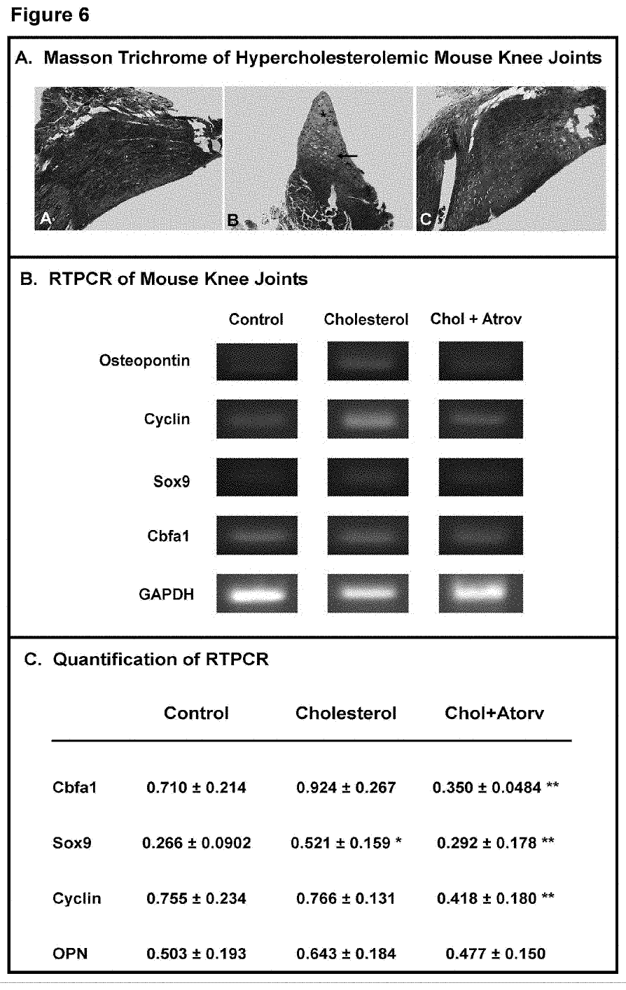

FIG. 6 is a phenotype characterization of mouse ApoE knee joints on a cholesterol diet with and without atorvastatin therapy Panel A-A Depicts Masson Trichrome for the ApoE control joints. Panel A-B depicts Masson Trichrome for the ApoE cholesterol joints. An arrow points to the chondrocyte and star over extracellular matrix. Panel AC depicts Masson Trichrome for the ApoE cholesterol+Atorvastatin joints. Referring now to FIG. 6 Panel B shows the RTPCR of Chondrocyte gene markers: Panel A. Composite of the Gene Expression for Cbfa1, Sox9, Cyclin and Osteopontin. Panel B. Demonstrates the quantification and statistical results of the data from the Semi Quantitative RTPCR. Referring now to FIG. 6 Panel C is a table indicating the quantification of the calcification markers of the three knee joints on a control diet, a cholesterol diet without atorvastatin and a cholesterol diet with atorvastatin therapy.

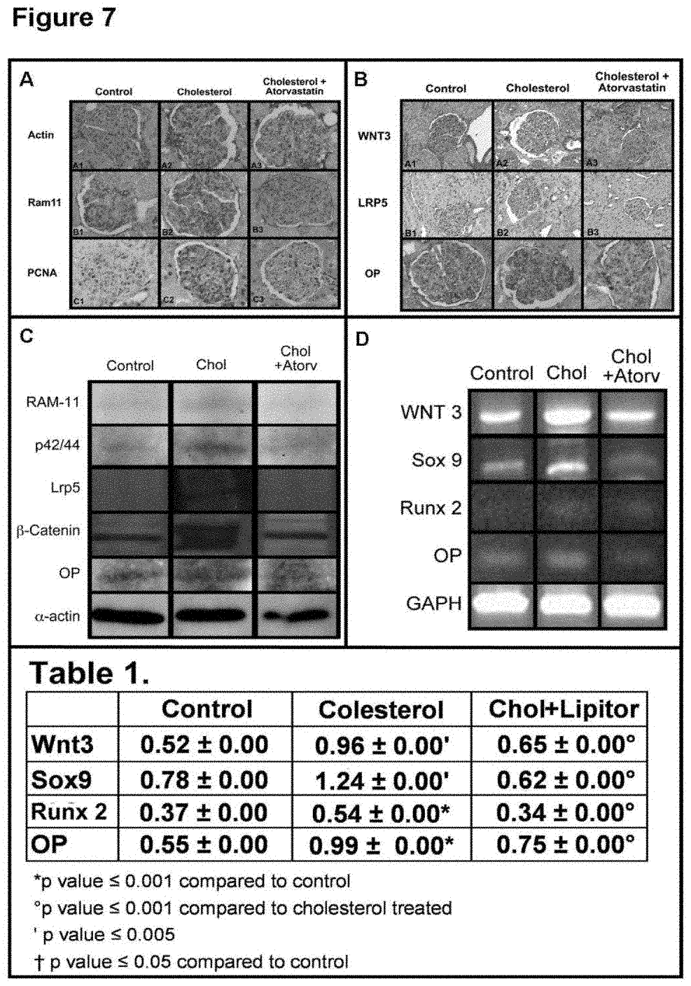

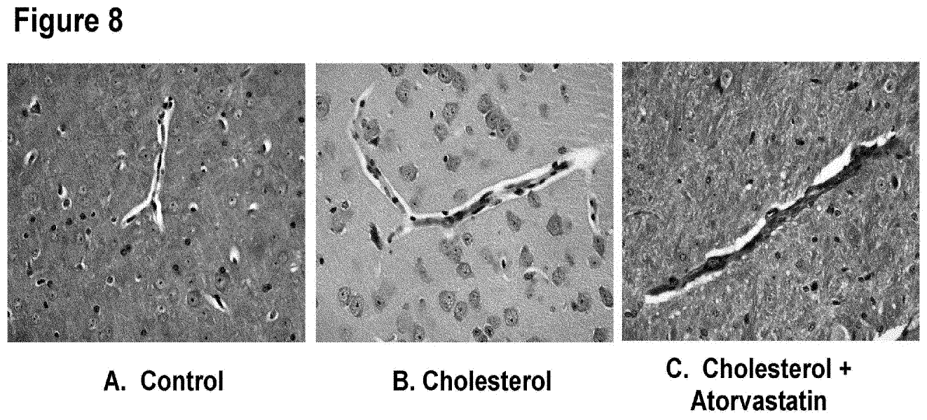

Referring now to FIG. 7, Panel A the phenotypic characterization of the kidneys of a rabbit on a cholesterol diet is shown. Panel B illustrates the immunohistochemistry of rabbit kidneys on the three different diets: control, cholesterol without atorvastatin and cholesterol with Atorvastatin. FIG. 7, Panel C shows RT PCR of calcification markers from rabbit kidneys on the three different diets: control, cholesterol without atorvastatin and cholesterol with Atorvastatin. FIG. 7, Panel D is a western blot analysis of calcification markers from rabbit kidneys on the three different diets: control, cholesterol without atorvastatin and cholesterol with Atorvastatin. FIG. 7, Table 1 represents the results of the semi-quantitative RTPCR for Wnt3, Sox9, Runx2 and OP. Western blots were performed for Ram 11, p42/44, Lrp5, beta-catenin, OP and alpha-actin. Creatinine values in control, cholesterol fed and atorvastatin treated rabbits were also assessed. As shown in Table 1 there is a statistically significant increase in gene expression and protein expression for the atherosclerotic and Wnt/Lrp5 markers in the cholesterol fed rabbit kidneys as compared to the control kidneys. FIG. 7, Panel A and FIG. 7, Panel B. demonstrate the alpha-actin, Ram11, PCNA, osteopontin, Wnt3a and Lrp5 effects within the glomerular vasculature. The control kidney in FIGS. 7 and 8, Panel A1-C1 demonstrates that there are few staining cells within the glomeruli for each of the different stains. Table 1 indicates a statistically significant increased protein expression for OP, BSP, RAM11, p42/44, b-catenin, Lrp5 in cholesterol fed rabbits in comparison to control animals. The atorvastatin treatment attenuated the effects exerted by cholesterol diet on gene and protein expression. Atorvastatin also improved the serum creatinine levels in the cholesterol treated rabbits.

FIG. 8 depicts the histology of the brains removed from the LDLR knockout mice on three different diets, control cholesterol and cholesterol+atorvastatin demonstrating improvement in the plaque lesions in the statin treated mouse brains.

The foregoing demonstrates that heart valves, spinal column, brain, kidneys, gastrointestinal tract and eyes express abnormal bone matrix proteins which cause heart valve calcification via the Lrp5-TIEG1 pathway. Specifically, the role of LDL activation of Lrp5 complex binding to Wnt, Frizzled, to activate β-catenin, TIEG1, and LEF1 in the nucleus. The activation of these signaling pathways is critical in ectopic abnormal bone formation in the human body in different organs.

Ectopic calcification can develop in heart valves, spinal column, brain, kidneys, gastrointestinal tract and eyes secondary to elevated LDL causing long term ectopic calcification and that inhibiting the level of LDL in the body can treat the disease mechanisms causing calcification including the use of PCSK9 inhibitors.

The further confirms the role of TIEG1 in osteogenic bone signaling including the role of TIEG1 in Wnt Signaling in osteogenic bone formation. TIEG1 is also upregulated and active in the calcifying aortic valve tissue from our mouse model.

What is claimed:

1. A method for inhibiting ectopic calcification in bodily organs caused by elevated LDL cholesterol levels, said method comprising:
administering an LDL lowering agent to a patient comprising a PCSK9 inhibitor selected from Evolocumab, Alirocumab, Praluent, Repatha Pushtonix, Repatha autoinjector, and combinations of the foregoing;
inhibiting the activation of Lrp5-TIEG pathways; and
slowing or reversing the progression of ectopic calcification caused by elevated LDL levels in the bloodstream, which activates the Lrp5 receptor.

2. The method of claim 1 further comprising inhibiting extracellular protein matrix production in an osteoblast cell originating from a native myofibroblast cell.

3. The method of claim 1 further comprising administering a subcutaneous dosage of the LDL lowering agent.

4. The method of claim 1 wherein said PCSK9 inhibitor is administered subcutaneously or intramuscularly.

5. The method of claim 1 wherein an initial dose of the PCSK9 inhibitor is from 0.25 mg/kg to 1.5 mg/kg.
